(19) United States
(12) Patent Application Publication
(10) Pub. No.: US 2023/0346394 A1
(43) Pub. Date: Nov. 2, 2023

(54) COMBINATION FEMORAL PREPARATION CUTTING BLOCKS FOR KNEE ARTHROPLASTY

(71) Applicant: NFS, LLC, Coronado, CA (US)

(72) Inventors: Steven N. Copp, Coronado, CA (US); Stacy F. Lang, La Jolla, CA (US)

(21) Appl. No.: 18/245,111

(22) PCT Filed: Sep. 15, 2021
(86) PCT No.: PCT/US2021/050501

§ 371 Date: Mar. 13, 2023

Related U.S. Application Data
(60) Provisional application No. 63/080,542, filed on Sep. 18, 2020.

(51) Int. Cl. A61B 17/17; A61B 17/15

(52) U.S. Cl. CPC A61B 17/1764; A61B 17/155

(57) ABSTRACT

A method of performing a knee arthroplasty procedure using no more than a single cutting block to prepare a femur can include securing a single cutting block with respect to the distal femur, resecting a portion of the anterior femur utilizing the cutting block, resecting a portion of the posterior condyle utilizing the cutting block, resecting a portion of the anterior chamfer utilizing the cutting block, resecting a portion of the posterior chamfer utilizing the cutting block, and resecting a portion of the sulcus utilizing the cutting block without removing the cutting block after resecting a portion of the posterior chamfer utilizing the cutting block. An integrated, combination cutting block for preparing a femur during a knee arthroplasty procedure can be provided. A low-profile cutting block for preparing a femur during a knee arthroplasty procedure can be provided.

### CROSS-REFERENCE TO RELATED APPLICATIONS

This application claims priority benefit to U.S. Provisional Patent Application No. 63/080,542, filed Sep. 18, 2020, the entirety of which is hereby incorporated by reference herein.

### BACKGROUND

Joint arthroplasty is a well-known surgical procedure by which a diseased and/or damaged natural joint is replaced by a prosthetic joint. For example, in a total knee arthroplasty surgical procedure, a patient's natural knee joint is partially or totally replaced by a prosthetic knee joint or knee prosthesis. A typical knee prosthesis includes a tibial tray, a femoral component, and a polymer insert or bearing positioned between the tibial tray and the femoral component. The tibial tray generally includes a plate having a stem extending distally therefrom, and the femoral component generally includes a pair of spaced apart condylar elements, which include surfaces that articulate with corresponding surfaces of the polymer bearing. The stem of the tibial tray is configured to be implanted in a surgically-prepared medullary canal of the patient's tibia, and the femoral component is configured to be coupled to a surgically-prepared distal end of a patient's femur. Such orthopedic procedures can employ a number of different cutting blocks including cutting guides to prepare the bone for a prosthetic implant. Improved cutting guides are needed, and described herein.

### SUMMARY

In some embodiments, a system, kit, and/or method can comprise, consist essentially of, or consist of any number of features as disclosed herein.

In some embodiments, disclosed herein is a method of performing a knee arthroplasty procedure using no more than (e.g., only) a single cutting block to prepare a femur, comprising any number of: securing a single cutting block with respect to the distal femur; resecting a portion of the anterior femur utilizing the cutting block; resecting a portion of the posterior condyle utilizing the cutting block; resecting a portion of the anterior chamfer utilizing the cutting block; resecting a portion of the posterior chamfer utilizing the cutting block; and/or resecting a portion of the sulcus utilizing the cutting block without removing the cutting block after resecting a portion of the posterior chamfer utilizing the cutting block.

In some embodiments, the method further comprises removing the cutting block after resecting a portion of the sulcus.

In some embodiments, the method further comprises positioning a knee prosthesis over the resected portions of the knee.

In some embodiments, resecting a portion of the sulcus comprises inserting a sulcus cutting tool in a sulcus cutting guide on an inferior surface of the cutting block.

In some embodiments, the sulcus cutting guide comprises a ramp extending superiorly and posteriorly from the inferior surface of the cutting block.

In some embodiments, the method further comprises inserting the sulcus cutting tool in a slot on the inferior surface.

In some embodiments, the method further comprises inserting a drill into a central aperture in the anterior cutting guide to confirm sizing.

In some embodiments, securing the single cutting block with respect to the distal femur comprises inserting a plurality of pins into posterior referencing pin holes in the single cutting block.

In some embodiments, disclosed herein is an integrated, combination cutting block for preparing a femur during a knee arthroplasty procedure, comprising any number of: an outer-facing surface, a bone-engaging surface, a superior surface, and an inferior surface; an anterior cutting guide; a posterior cutting guide; a plurality of chamfer cutting guides; and/or a sulcus cutting guide inferior to the anterior, posterior, and chamfer cutting guides.

In some embodiments, the sulcus cutting guide comprises a guide ramp that extends from the inferior surface posteriorly and superiorly to at least the outer-facing surface, and at an angle with respect to a plane of the outer-facing surface.

In some embodiments, the sulcus cutting guide is a captured cutting guide.

In some embodiments, the sulcus cutting guide comprising an entrance slot on the inferior surface and an exit slot on the bone-engaging surface.

In some embodiments, the sulcus cutting guide is a non-captured cutting guide.

In some embodiments, the angle is between about 15 degrees and about 35 degrees, such as about 25 degrees.

In some embodiments, disclosed herein is a low-profile cutting block for preparing a femur during a knee arthroplasty procedure, comprising any number of: an outer-facing surface, a bone-engaging surface, a superior surface, and an inferior surface; an anterior cutting guide; a posterior cutting guide; a plurality of chamfer cutting guides; and/or a plurality of laterally spaced apart cut-out portions between the anterior cutting guide and plurality of chamfer cutting guides, each laterally spaced apart cut-out portions spaced apart by a bridge.

In some embodiments, the anterior cutting guide comprises a slot, and the slot comprises a drill aperture comprising a diameter greater than the width of the slot.

In some embodiments, a surface area of the cut-out portions is between about 5% and about 25% of the total surface area of the outer-facing surface.

In some embodiments, a cutting block can further comprise a sulcus cutting guide on the cutting block positioned inferior to the anterior, posterior, and chamfer cutting guides.

In some embodiments, a cutting block does not comprise anterior referencing pin holes.

### BRIEF DESCRIPTION OF THE DRAWINGS

FIG. 1A is a perspective view of an orthopedic surgical instrument;

FIG. 1B is an elevation view showing the outer surface of the orthopedic surgical instrument of FIG. 1A;

FIG. 1C is an elevation view of the bone-engaging surface of the orthopedic surgical instrument of FIG. 1A;

FIG. 1D is a cross section view of the orthopedic surgical instrument, taken along the line 4-4 of FIG. 1B, as viewed in the direction of the arrows;

FIG. 1E is a perspective view showing a pair of fixation pins secured to a distal end of a patient's femur;

FIG. 1F is a view similar to FIG. 1E, but showing the orthopedic surgical instrument of FIG. 1A being used to perform the anterior and posterior cuts on the distal end of the patient's femur; and

FIG. 1G is a view similar to FIG. 1F, but showing the orthopedic surgical instrument of FIG. 1A being used to perform the chamfer cuts on the distal end of the patient's femur.

FIG. 1H illustrates a perspective view of another embodiment of an anterior/posterior chamfer cutting block, according to some embodiments, that does not include any integrated sulcus cutting features.

FIG. 1I illustrates the cutting block of FIG. 1H, with a posterior perspective view encircled, illustrating a plurality of pins being inserted into the femur, along with posterior alignment slots as shown.

FIG. 1J illustrates an anterior view 4 in 1 cutting block attached on the knee, with posterior referencing pin holes inserted for anchoring.

FIG. 1K illustrates a perspective view of the 4 in 1 cutting block of FIG. 1J.

FIG. 1L illustrates a discrete second cutting block configured to perform a single cut (a sulcus cut).

FIG. 1M is a side view of the discrete cutting block of FIG. 1L.

FIG. 1N illustrates a blade view of the cutting block of FIGS. 1L and 1M, showing the cutting tool after being advanced on the sulcus cutting guide ramp.

FIG. 2A illustrates an inferior perspective view of a combination 5-in-1 cutting block 200, according to some embodiments.

FIG. 2B illustrates a posterior perspective view of the bone-engaging surface 220 of the cutting block of FIG. 2A, also illustrating the exit slot 203 of the sulcus cutting slot 207 and connected via a ramp guide 205 which can be partially or fully enclosed within the cutting block.

FIG. 2C illustrates another view of the inferior surface of the cutting block, including the entrance sulcus cutting slot.

FIG. 2D-2D1 illustrate a low-profile cutting block can have a reduced footprint in comparison to other cutting blocks as described and illustrated, for example, herein.

FIGS. 2E and 2F include anterior/inferior angled and posterior angled respective perspective views of a combination cutting block that can include any number of features as described elsewhere herein.

FIG. 2G illustrates a cross-sectional view of a cutting block that could include, for example, features of the cutting block of FIGS. 2A-2B.

FIGS. 3A-3G illustrate sequences of a method of performing a total knee arthroplasty utilizing a combination 5-in-1 cutting block, according to some embodiments of the invention.

FIG. 3H illustrates a discrete sulcus cutting block (upper left); 4-in-1 cutting block (upper right), and a low-profile combination 5-in-1 cutting block (bottom).

### DETAILED DESCRIPTION

Traditional total knee arthroplasty (TKA) can be performed with systems including but not limited to, for example, the ATTUNE® system by DePuy Synthes Companies (Warsaw, IN). The procedure can be initiated with surgical exposure of the knee, most commonly through a medial parapatellar arthrotomy. With adequate exposure, the intramedullary canal of the femur is opened. An intramedullary guide is placed with a distal femoral cutting block attached at a defined angle. The distal femoral cutting block is then fixed to the distal femur, e.g., about 9 mm from the anatomic end of the native femur. The distal femoral cut is then performed. With the resection of the distal femur completed, the anterior/posterior size of the resected distal femur can be measured and the rotational axis of the femur calculated. Pins can then be placed in the distal femur in the selected rotation position for placement of the 4 in 1 cutting block for further preparation of the femur. The 4 in 1 cutting block selected is based on the anterior-posterior size of the femur previously measured and will match the eventual size of the distal femoral component of the system. The selected 4 in 1 block is secured with pins into the distal femur through the block. The original pins for block placement are removed. The distal aspect of the femur is then shaped for the eventual placement of the prosthesis with anterior femoral, posterior condylar, and anterior and posterior chamfer cuts. The first cutting block and bone fragments are removed. Next, a separate, second cutting block is placed over the femur and secured with two pins. This second block is a guide for additional bone removal, known as a sulcus cut needed to provide a space for this portion of the TKA prosthesis. After completion of the cut, the block and bone fragment are removed, and the prosthesis can be installed over the resected portion of the knee. Procedures can be performed manually by an operator such as an orthopedic surgeon, for example, or fully or partially utilizing robotics and/or computer assistance in other embodiments.

In some embodiments, as described herein any number of features of the first cutting block and second cutting block can be combined into a single, integrated cutting block.

In some embodiments, a 4-in-1, 5-in-1, or other cutting block can include any number of features disclosed, for example, in U.S. Pub. No. 2014/0257306 to Edwards et al., which is hereby incorporated by reference in its entirety. While some embodiments of cutting blocks described herein can be configured for one-time use, the blocks can be modified (e.g., with different materials) to be configured for multiple uses.

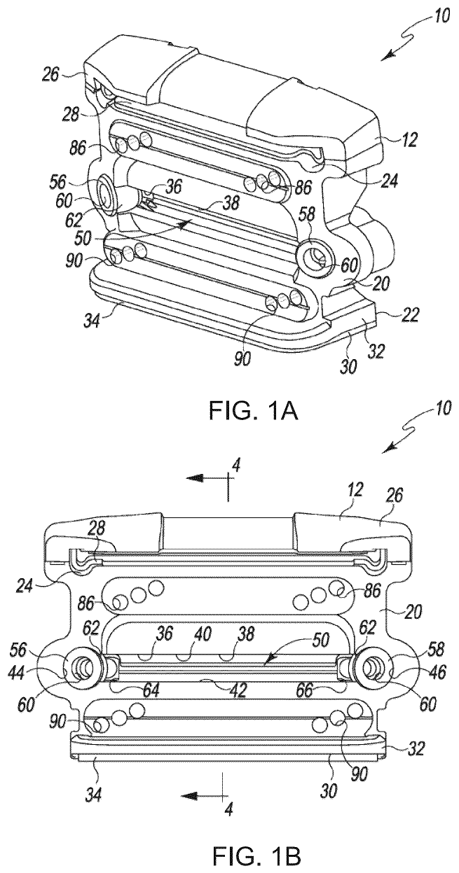

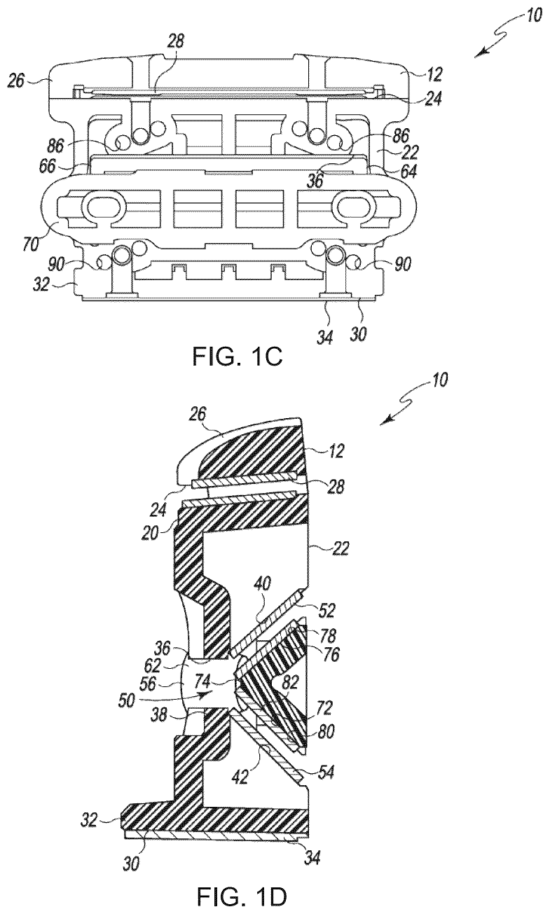

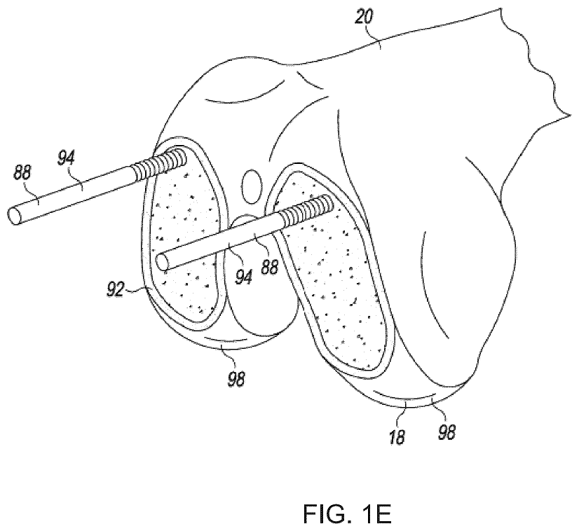

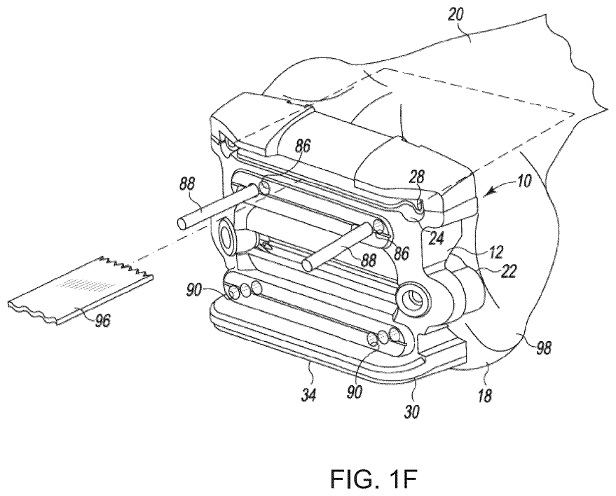

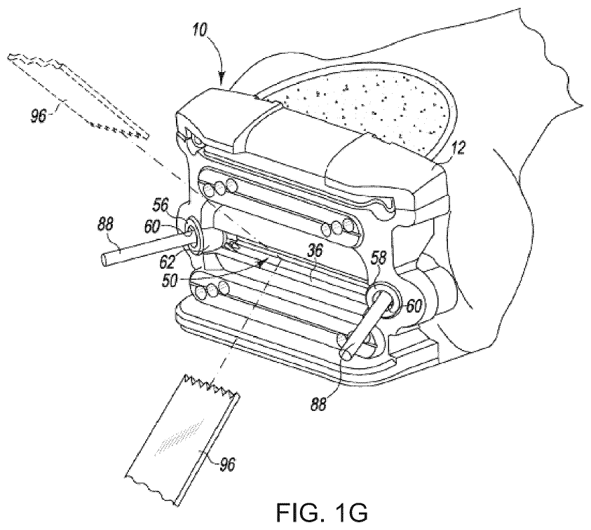

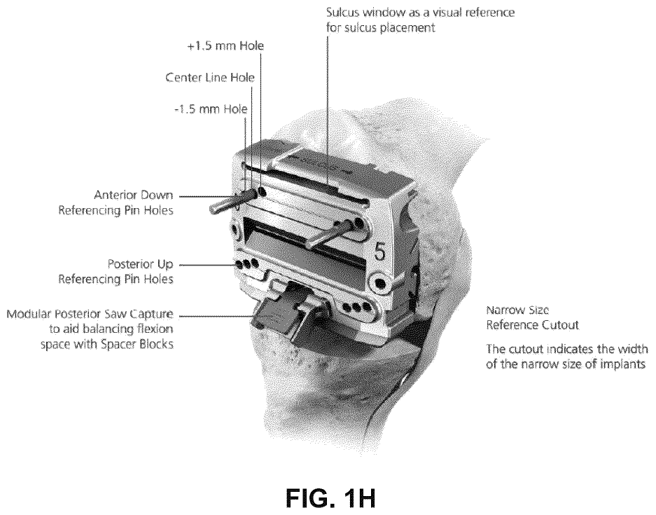

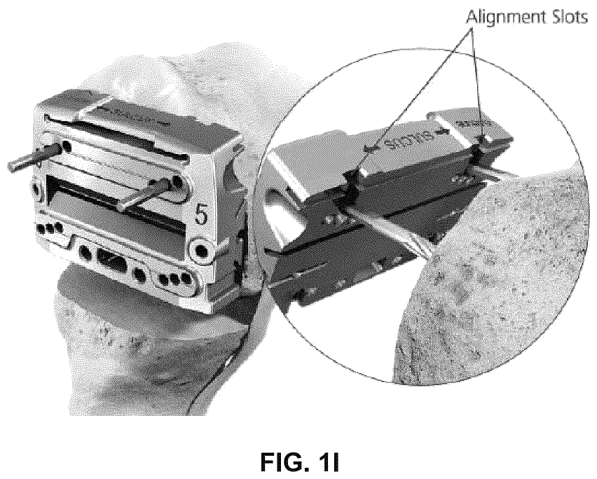

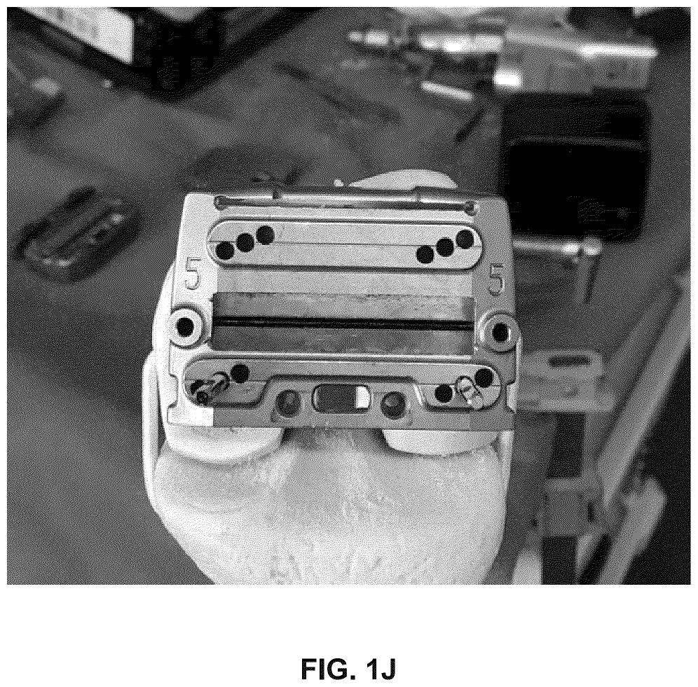

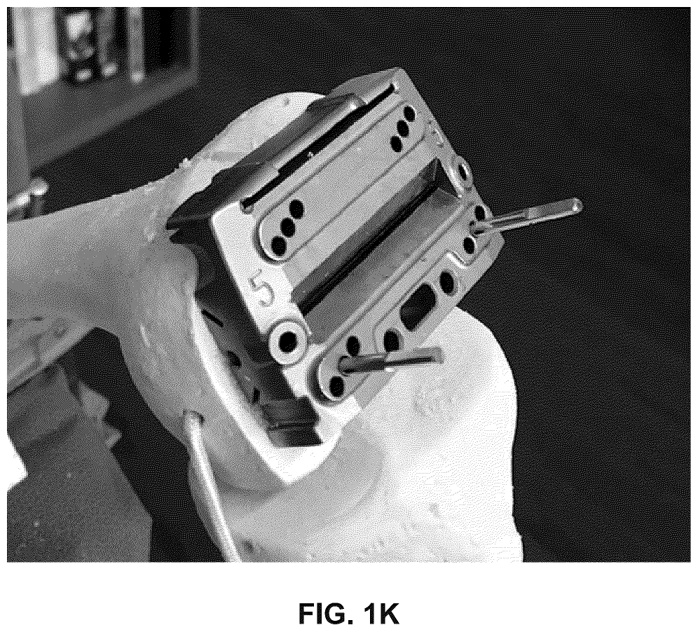

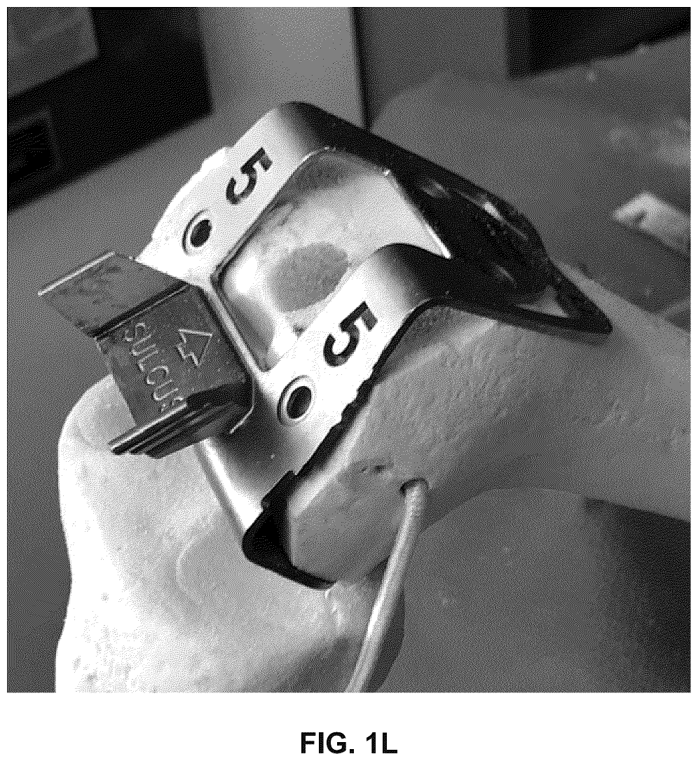

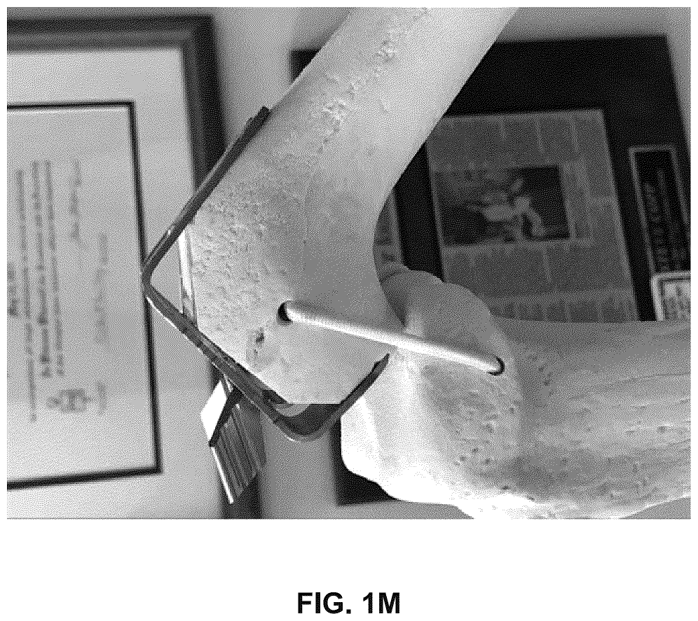

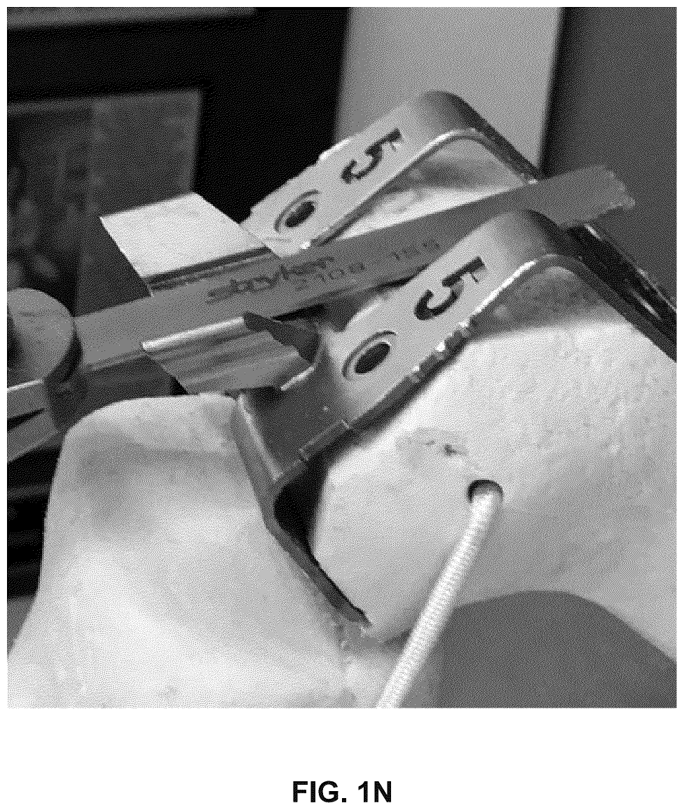

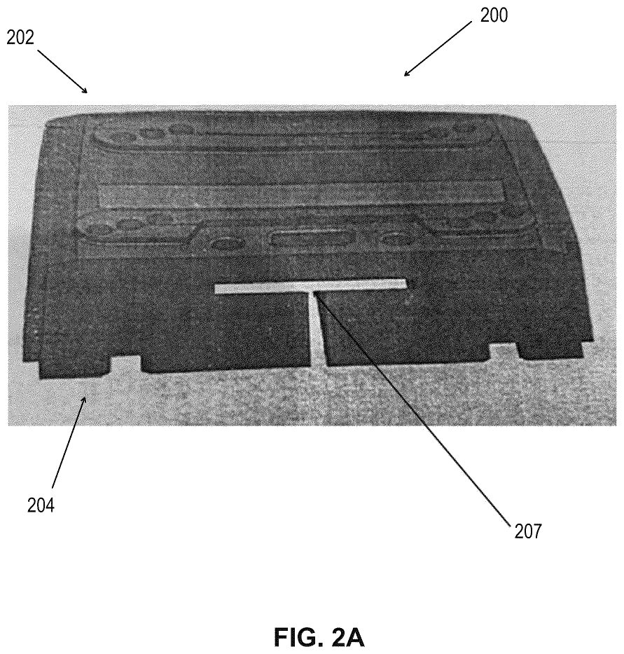

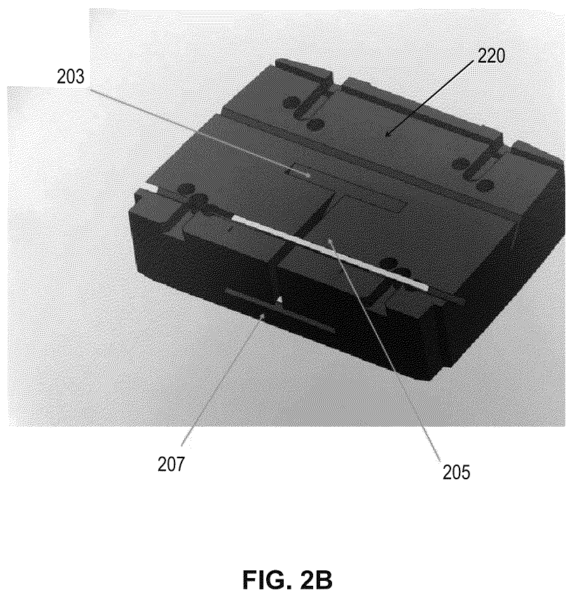

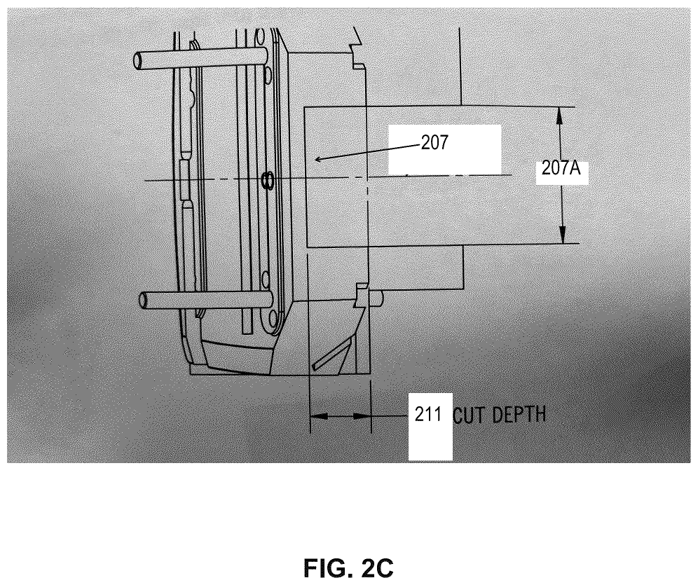

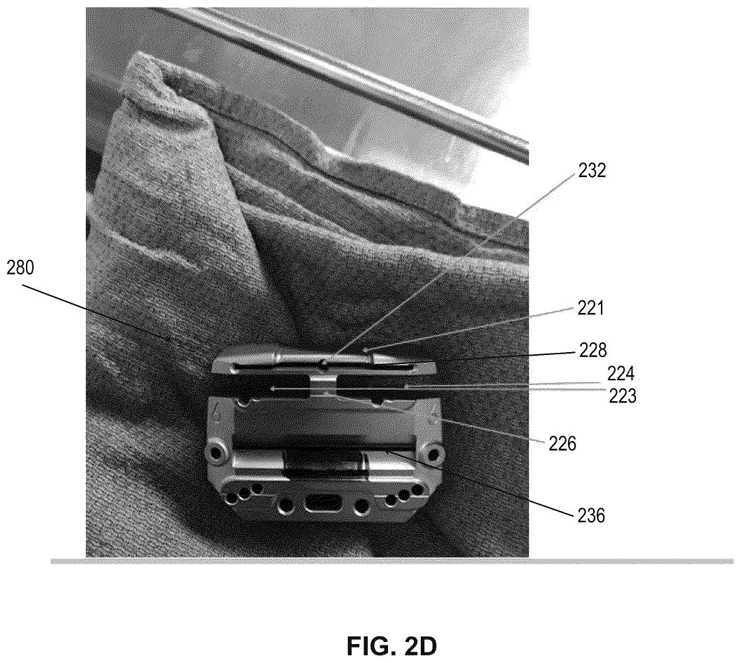

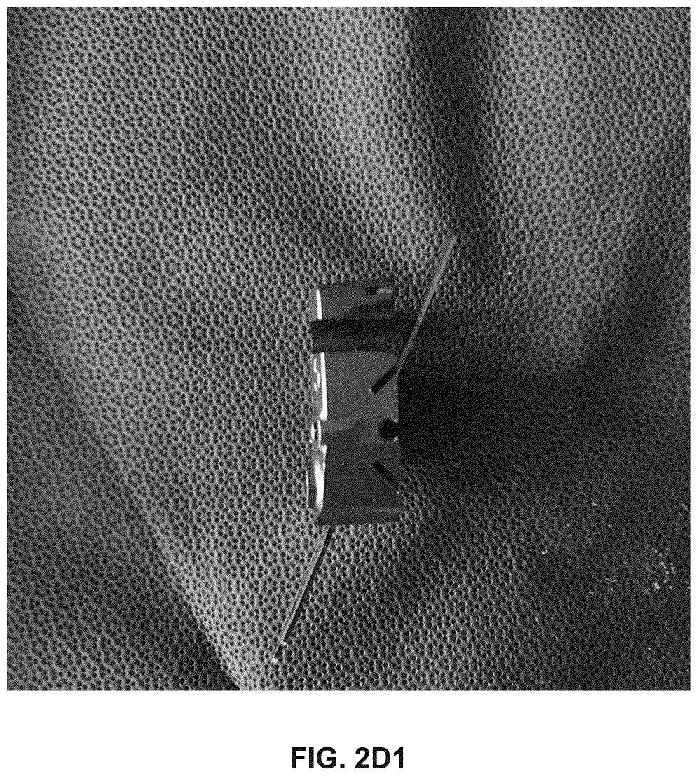

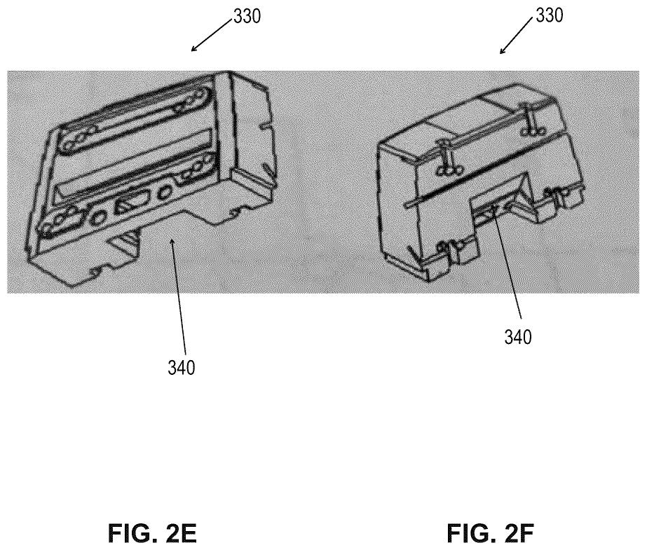

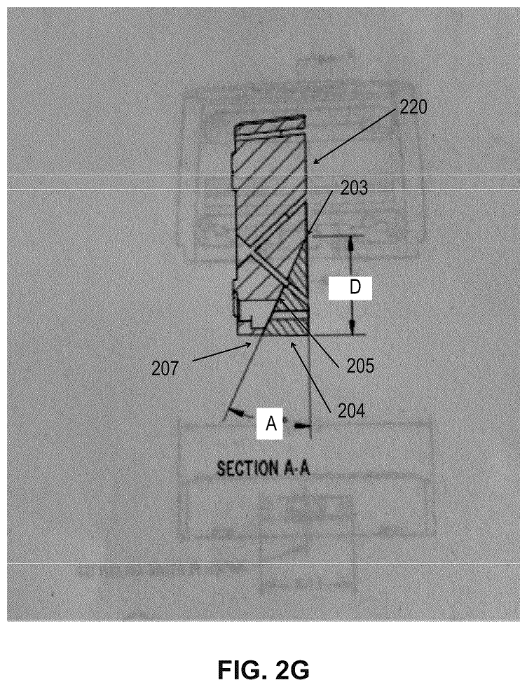

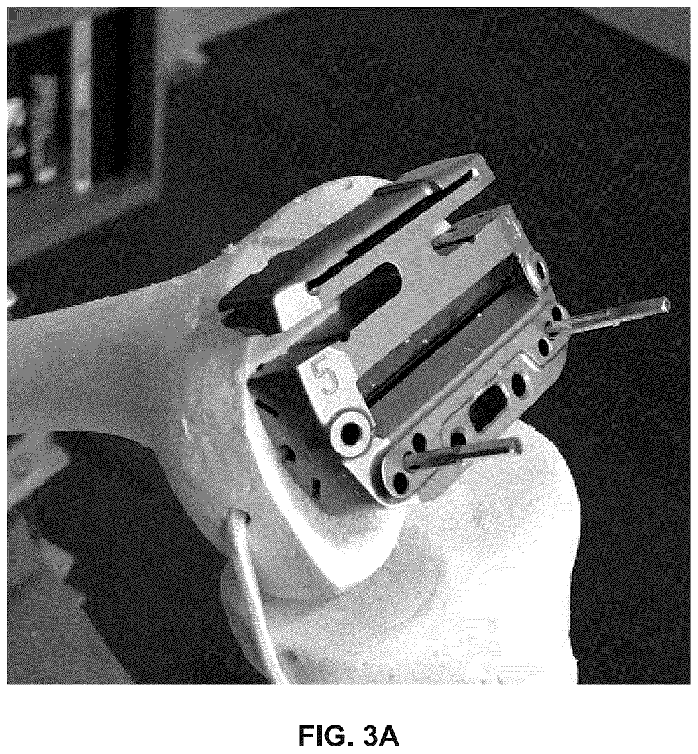

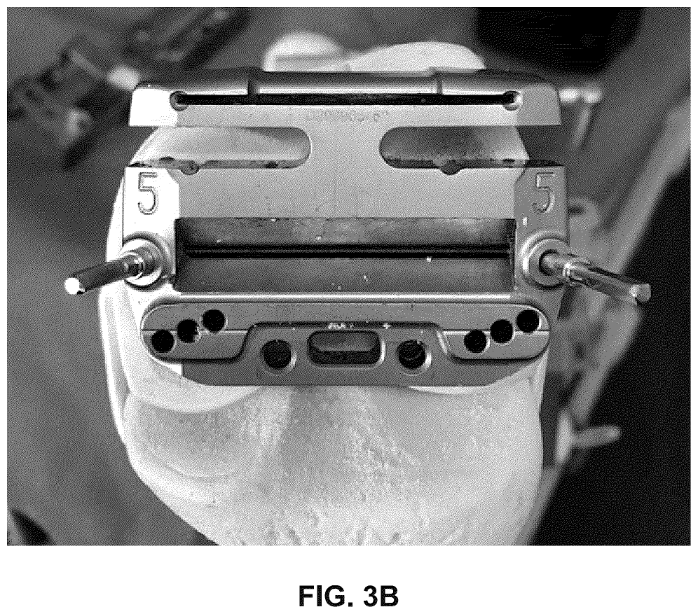

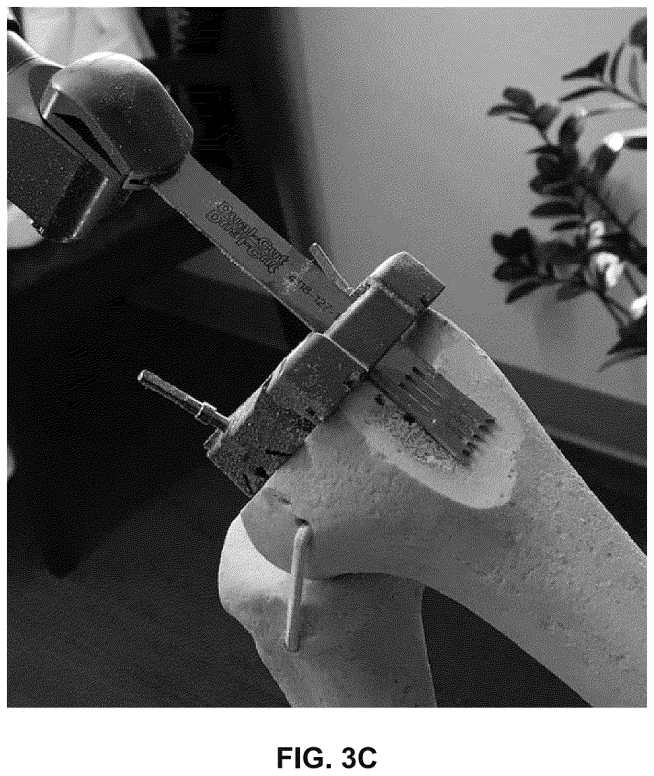

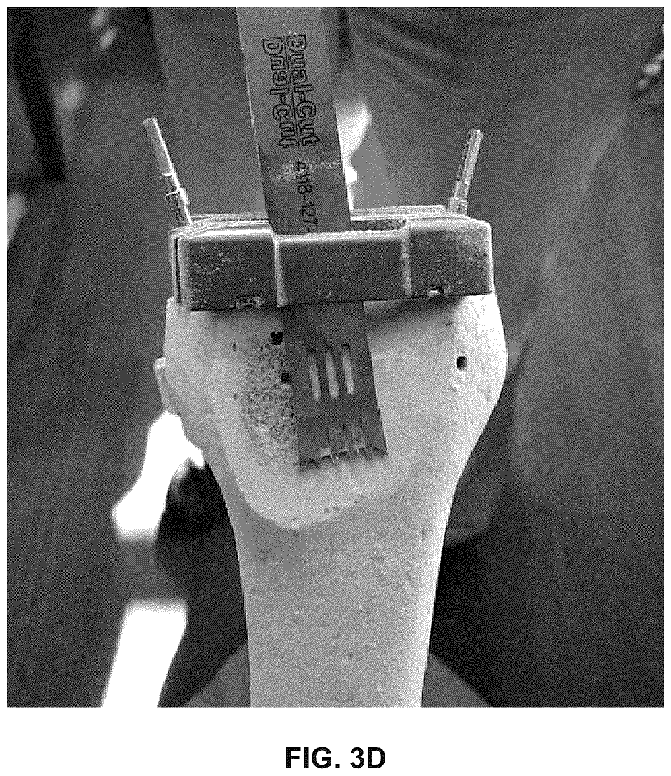

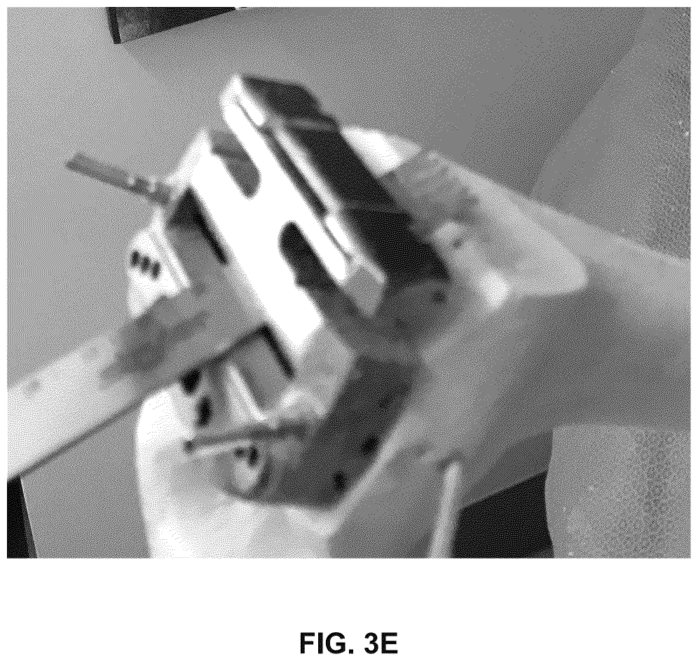

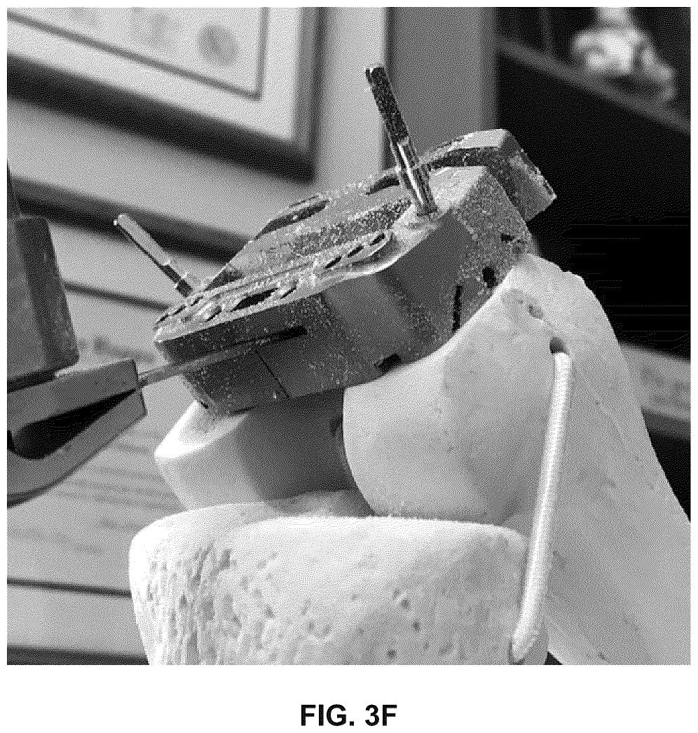

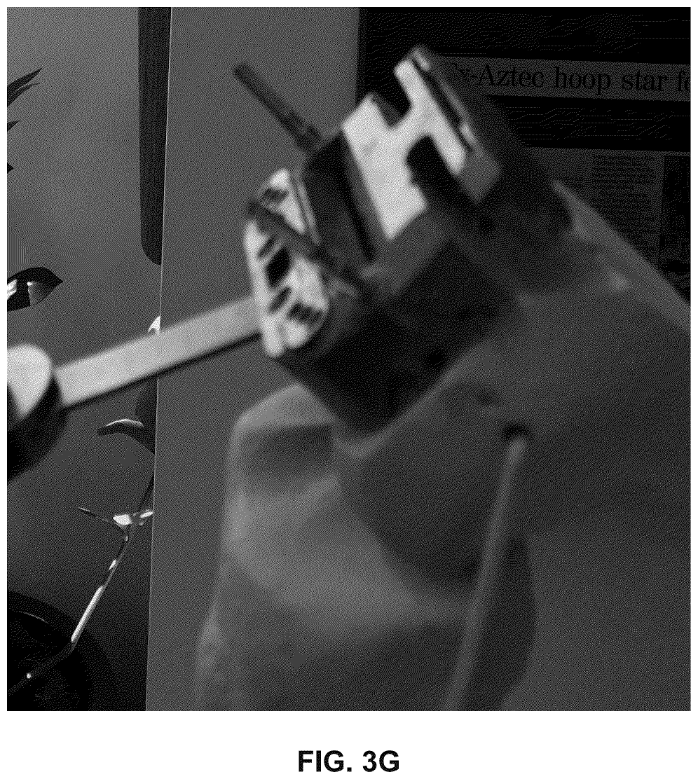

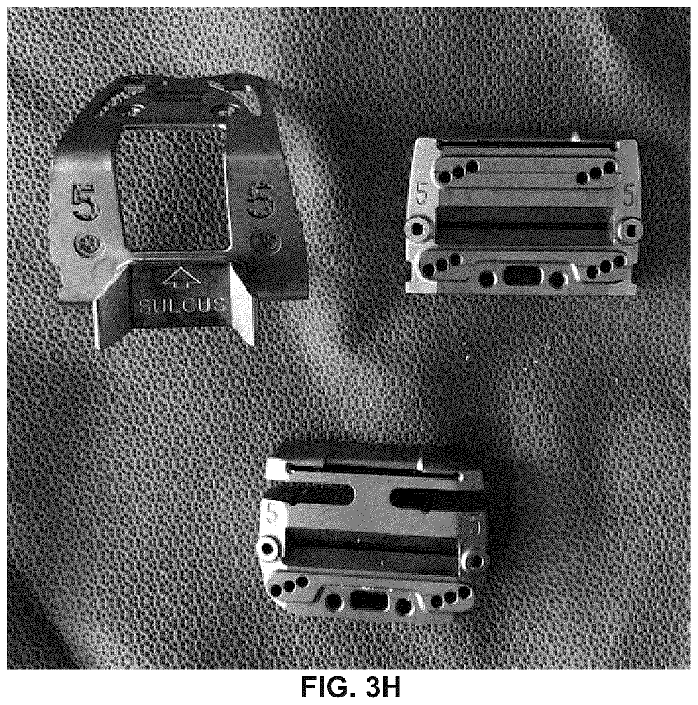

FIGS. 1A-1D illustrate embodiments of an orthopedic surgical instrument 10 is shown, which can be configured to be disposed of after use in a single orthopedic procedure, or adapted for re-use and able to be autoclaved or otherwise re-sterilized in some embodiments. In the illustrative embodiment described herein, the orthopedic surgical instrument is embodied as a 4-in-1 cutting block 12 for use in the surgical preparation of the patient's distal femur during a knee replacement procedure, such as a total knee arthroplasty, for example. As will be discussed below in greater detail, a 4-in-1 cutting block 12 is used to perform four cuts on the patient's distal femur with the same block—an anterior cut, a posterior cut, and two chamfer cuts. Such cutting blocks could also incorporate any number of features of cutting blocks described and illustrated, for example, in FIGS. 2A-3J below, including configuring the blocks as 5-in-1 cutting blocks.

As a single use or multiple use instrument, the 4-in-1 cutting block 12 may be formed from a metal, and/or polymeric materials such as, for example, polyamide, polyphenylsulfone, or polyketone depending on the desired indication. In some embodiments, the surfaces used to guide surgical instruments, such as cutting guide surfaces for guiding bone saws and bushings for guiding drills and surgical pins, are formed from a metallic material such as, for example, steel, titanium alloy, or cobalt chromium alloy. Such use of metallic components or “inserts” prevents the surgical tools from coming into contact with the polymeric materials of the block's body. In some embodiments, a cutting block does not include any polymeric materials.

The metallic components described herein, if present, can be secured to the 4-in-1 cutting block in a number of different manners. For example, the metallic components may be overmolded to the polymer cutting block or otherwise secured to it as part of the molding process of the block. The metallic components may also be welded to the cutting block or secured to it with an adhesive. Other methods of securing the metallic components may also be employed. In some embodiments, the cutting block is made of a metallic material without any polymers.

The 4-in-1 cutting block 12 includes an outer surface 20 and a bone-engaging surface 22 positioned opposite the outer surface 20. The 4-in-1 cutting block 12 can include an anterior cutting slot 24 formed near its anterior end 26. The anterior cutting slot 24 can be an elongated slot extending in the medial/lateral direction. The anterior cutting slot 24 can extends through the entire thickness of the 4-in-1 cutting block 12. For example, the anterior cutting slot 24 extends from the cutting block's outer surface 20 to its bone-engaging surface 22 thereby being open to both surfaces. An anterior cutting guide 28 can be secured within the anterior cutting slot 24 of the 4-in-1 cutting block 12. The anterior cutting guide 28 lines the anterior cutting slot 24 and is embodied as a captured cutting guide (i.e., it is closed on all sides so as to capture a saw blade therein), although the cutting block 12 and the cutting guide 28 may alternatively be embodied as a non-captured cutting guide. The anterior cutting guide 28 is sized and shaped to receive the blade (as shown in FIG. 1F) of a surgical saw or other cutting instrument and orient the blade to resect the anterior surface of the patient's femur during an orthopedic surgical procedure.

The cutting block 12 can include a posterior cutting surface 30 formed near its posterior end 32. The posterior cutting surface 30 can be an elongated surface extending generally in the medial/lateral direction. The posterior cutting surface 30 can extend the entire thickness of the 4-in-1 cutting block 12—that is, it extends from the cutting block's outer surface 20 to its bone-engaging surface 22. A posterior cutting guide 34 can be secured to the posterior cutting surface 30 of the 4-in-1 cutting block 12. The posterior cutting guide 34 is sized and shaped to support and guide the blade (as shown in FIG. 1F) of a surgical saw or other cutting instrument and orient the blade to resect the posterior surface of the patient's femur during an orthopedic surgical procedure. In the illustrative embodiment described herein, the posterior cutting guide 34 is embodied as a non-captured cutting guide, although it may alternatively be embodied as a captured cutting guide.

The cutting block 12 has a chamfer cutting slot 36 formed near its middle. Specifically, the chamfer cutting slot 36 is located posteriorly of the anterior cutting slot 24 and anteriorly of the posterior cutting surface 30. The chamfer cutting slot 36 is an elongated slot extending in the medial/lateral direction. The chamfer cutting slot 36 extends through the entire thickness of the cutting block 12—that is, it extends from the cutting block's outer surface 20 to its bone-engaging surface 22 and, as a result, opens to both surfaces. The chamfer cutting slot 36 is defined by a sidewall 38 of the 4-in-1 cutting block that includes an anterior edge 40 extending in the medial/lateral direction and a posterior edge 42 that is spaced apart from the anterior edge 40 and likewise extends in the medial/lateral direction. The chamfer cutting slot 36 can include enlarged rounded medial and lateral ends. In particular the medial edge 44 of the sidewall 38 defining the chamfer cutting slot 36 can be cylindrical in shape (e.g., circular when viewed in the front elevation of FIG. 1B) and can include a diameter that is larger than the anterior-posterior width of the slot 36 (e.g., the distance between anterior edge 40 and the posterior edge 42 of the sidewall 38). On the opposite end of the chamfer cutting slot 36, the lateral edge 46 of the sidewall 38 can be identical in shape and size. In the illustrative embodiment described herein, the chamber cutting slot 36 can take on the form of two cylinders spaced at opposite medial and lateral ends connected by an elongated planar slot, although other geometries are also possible.

As can be seen in FIGS. 1A, 1B, and 1D, a metallic captured chamfer cutting guide assembly 50 is positioned in the chamfer cutting slot 36. The chamfer cutting guide assembly 50 includes a metallic planar cutting guide 52 secured to the anterior edge 40 of the sidewall 38 defining the anterior side of the chamfer cutting slot 36, along with a metallic planar cutting guide 54 secured to the posterior edge 42 of the sidewall 38 defining the posterior side of the chamfer cutting slot 36. As can be seen best in FIG. 1D, the planar cutting guides 52, 54 are spaced apart from one another in the anterior/posterior direction and are arranged at an oblique angle relative to one another. The longitudinal axis of the planar cutting guides 52, 54 can extend in the medial/lateral direction.

The medial and lateral ends of the captured chamfer cutting guide assembly 50 can be defined by a pair of bushings 56, 58, that can be metallic in some cases. In some embodiments, the metallic bushing 56 is positioned in the enlarged cylindrically-shaped medial end of the chamfer cutting slot 36—that is, the metallic bushing 56 is secured in contact with the medial edge 44 of the sidewall 38 defining the medial end of the chamfer cutting slot 36. The metallic bushing 58 can be positioned in the enlarged cylindrically-shaped lateral end of the chamfer cutting slot 36—that is, the metallic bushing 58 is secured in contact with the lateral edge 46 of the sidewall 38 defining the lateral end of the chamfer cutting slot 36.

The metallic bushings 56, 58 can be cylindrically-shaped in some embodiments and have an elongated bore 60 extending therethrough. The elongated bore 60 can be sized to receive a fixation or guide pin for pinning the cutting block to the patient's distal femur (see FIG. 1G), and, optionally, a drill such that the patient's femur may be pre-drilled prior to installation of the guide pins if the surgeon so desires. The metallic bushings 56, 58 can be identical or different in shape and include an annularly-shaped outer surface 62. As can be seen in FIG. 1B, the outer surface 62 of the metallic bushing 56 can be positioned at the medial end 64 of the planar cutting guides 52, 54, with the outer surface 62 of the metallic busing 58 being positioned at the lateral end 66 of the planar cutting guides 52, 54. In the illustrative embodiment described herein, the outer surface 62 of the metallic bushings 56, 58 is slightly spaced apart from the respective medial end 64 and lateral end 66 of the planar cutting guides 52, 54 (e.g., the bushings 56, 58 are not positioned in contact with the planar cutting guides 52, 54). Such spacing allows for capture of the blade of a bone saw, while also providing for relief from overly tight tolerances in the manufacturing process. However, in another illustrative embodiment, the bushings 56, 58 are positioned in contact with the respective medial end 64 and lateral end 66 of the planar cutting guides 52, 54.

As can be seen best in FIGS. 1A and 1D, the cutting block 12 can include a wedge component 70 secured to its bone-engaging surface 22. Like the cutting block 12, the wedge component 70 can include polymeric, metallic, or other materials. The wedge component 70 has a wedge-shaped cutting surface 72. The “leading” edge 74 of the wedge-shaped cutting surface 72 extends into the chamfer cutting slot 36. A planar cutting guide 76 can be secured to the anterior surface 78 of the wedge-shaped cutting surface 72, with a planar cutting guide 80 being secured to the posterior surface 82 of the wedge-shaped cutting surface 72. Like the cutting guides 52, 54, the planar cutting guides 76, 80 can be spaced apart from one another and are arranged at an oblique angle relative to one another. The longitudinal axis of the planar cutting guides 76, 80 can generally extend in the medial/lateral direction. As can be seen in FIG. 1D, the cutting guide 76 is spaced apart from, and parallel to, the cutting guide 52, with the cutting guide 80 being spaced apart from, and parallel to, the cutting guide 54. In such a way, the cutting guides 52, 76 cooperate to guide a saw blade during performance of the anterior chamfer cut, with the cutting guides 54, 80 cooperating to guide the saw blade during performance of the posterior chamfer cut (see FIG. 1G).

As with the planar cutting guides 52, 54, the outer surface 62 of the bushing 56 can be positioned at the medial end of the planar cutting guides 76, 80, with the outer surface 62 of the bushing 58 being positioned at the lateral end of the planar cutting guides 76, 80. In the illustrative embodiment described herein, the outer surface 62 of the bushings 56, 58 is slightly spaced apart from the respective medial end and lateral end of the planar cutting guides 76, 80 (e.g., the bushings 56, 58 are not positioned in contact with the planar cutting guides 76, 80). Such spacing allows for capture of the blade of a bone saw, while also providing for relief from overly tight tolerances in the manufacturing process. However, in another illustrative embodiment, the bushings 56, 58 are positioned in contact with the respective medial end and lateral end of the planar cutting guides 76, 80.

The cutting block 12 can include a plurality of guide holes 86 defined therein that are sized to receive a pair of fixation or guide pins 88 (see FIG. 1F). The holes 86 can be positioned between the anterior cutting slot 24 and the chamfer cutting slot 36 and extend between the outer surface 20 and the bone-engaging surface 22 of the cutting block 12. The holes 86 can be arranged in a staggered pattern to permit the surgeon to change the position of the cutting block 12 on the patient's femur without having to remove the fixation pins 88, as described in greater detail below.

The cutting block 12 can also include another plurality of guide holes 90 positioned between the chamfer cutting 36 and the posterior cutting surface 30. Each guide hole 90 can be sized to receive one of the fixation pins 88 in a similar manner to the guide holes 86 and thereby extends between the outer surface 20 and the bone-engaging surface 22 of the cutting block 12. Like the guide holes 86, the guide holes 90 are arranged in a staggered pattern to permit the surgeon to change the position of the cutting block 12 on the patient's femur without having to remove the fixation pins 88.

In operation, the surgeon may utilize the orthopedic surgical instrument 10 to prepare the distal end 18 of the patient's femur 20 to receive a prosthetic femoral component. To do so, the surgeon may secure the cutting block 12 to the patient's femur 20 and thereafter use the metallic cutting guides of the cutting block 12 to guide a cutting saw blade in making a series of four resections of the distal end 18 of the patient's femur 20.

During an orthopedic surgical procedure, the surgeon may first resect the distal end 18 of the patient's femur 20 to create a surgically-prepared distal surface 92. The surgeon may then secure a pair of fixation pins 88 to the surgically-prepared distal surface 92 of the patient's femur 20, as shown in FIG. 1E. To do so, the surgeon may size the patient's femur 20 for the prosthetic femoral component and set the femoral rotation. After sizing the femoral component and setting the rotation, the surgeon may attach the fixation pins 88 to the surgically-prepared distal surface 92 of the patient's femur 20.

After attaching the fixation pins 88, the surgeon may position the cutting block 12 on the surgically-prepared distal surface 92 of the patient's femur 20. To do so, the surgeon may align the shafts 94 of the fixation pins 88 with a pair of the guide holes 86 of the cutting block 12. The surgeon may then advance the cutting block 12 over the shafts 94 in a direction toward the surgically-prepared distal surface 92 of the patient's femur 20. The bone-engaging surface 22 of the cutting block 12 contacts the surgically-prepared distal surface 92 when the instrument 10 is positioned on the distal end 18 of the patient's femur 20, as shown in FIG. 1G. If the surgeon desires to relocate the cutting block 12, the surgeon may utilize another combination of guide holes 86 to change the position of the cutting block 12 on the patient's femur 20. If additional fixation is necessary, the surgeon may insert additional fixation pins 88 through the guide holes 90 defined in the cutting block 12.

Once installed in such a manner, the surgeon may use the cutting block 12 to make a number of resections of the distal end 18 of the patient's femur 20. For example, as shown in FIG. 1G, the anterior cutting guide 28 defines a resection plane that extends through the distal end 18 of the patient's femur 20. The surgeon may advance a cutting tool, such as, for example, a surgical cutting saw 96 through the anterior cutting guide 28 to engage the patient's femur 20 and operate the surgical saw 96 to surgically prepare an anterior surface of the patient's femur 20 to receive the prosthetic femoral component. The surgeon may similarly use the posterior cutting guide 34 to resect the posterior condyles 98 of the patient's femur 20 and surgically prepare the posterior surfaces of the patient's femur 20 to receive the prosthetic femoral component.

As shown in FIG. 1G, the surgeon may also use the captured chamfer cutting guide assembly 50 in cooperation with the planar cutting guides 76, 80 of the wedge component 70 to make chamfer cuts on the patient's femur 20. To do so, the surgeon may first insert fixation pins 88 through the elongated bores 60 of the bushings 56, 58 of the chamfer cutting guides assembly 50. The surgeon may then remove any fixation pins 88 from the guide holes 86, 90 since fixation pins 88 positioned in the guide holes 86, 90 would disrupt the chamfer cutting process. The surgeon may then advance the surgical cutting saw 96 through opening between the cutting guides 52, 76 to guide the saw 96 during performance of the anterior chamfer cut (as shown in solid lines in FIG. 1G), and thereafter through the opening between the cutting guides 54, 80 to guide the saw 96 during performance of the posterior chamfer cut (as shown in phantom lines in FIG. 1G).

During performance of such chamfer cuts, the cutting guides 52, 54 function as a saw stop to prevent the saw 96 from engaging the body of the cutting block 12 that defines the anterior and posterior edges of the chamfer cutting slot 36. Similarly, the outer surfaces 62 of the bushings 56, 58 function as a saw stop to prevent the saw from engaging the polymeric body of the cutting block 12 that defines the medial and lateral edges of the chamfer cutting slot 36. Likewise, the wedge component's cutting guides 76, 80 function as a saw stop to prevent the saw 96 from engaging the wedge-shaped cutting surface 72 of the wedge component 12.

FIG. 1H illustrates a perspective view of another embodiment of an anterior/posterior chamfer cutting block, according to some embodiments, that does not include any integrated sulcus cutting features. The cutting block can include, for example, a sulcus window as a visual reference for sulcus placement, a plurality of anterior referencing pin holes (as illustrated, 3 laterally-spaced apart holes to allow the surgeon the opportunity for options in potential pin placement, including offset placement, with center line, +1.5 mm, and −1.5 mm holes as shown), a plurality of posterior referencing pin holes (as illustrated, 3 laterally-spaced apart holes to allow the surgeon the opportunity for options in potential pin placement, including offset placement), a modular posterior saw capture to assist balancing flexion space with spacer blocks, and a reference cutout indicating the width of the narrow size of the implants. Also illustrated are divergent pin holes on the medial and lateral aspects of the cutting block. The cutting block can also include slots configured to allow a cutting instrument to pass through to allow for anterior and posterior femur cuts, as well as angled ramps to allow for anterior chamfer (if posterior referencing) or posterior chamfer (if anterior referencing) cuts.

FIG. 1I illustrates the cutting block of FIG. 1H, with a posterior perspective view encircled, illustrating a plurality of pins being inserted into the femur, along with posterior alignment slots as shown.

FIG. 1J illustrates an anterior view 4 in 1 cutting block attached on the knee, with posterior referencing pin holes inserted for anchoring. FIG. 1K illustrates a perspective view of the 4 in 1 cutting block of FIG. 1J.

FIG. 1L illustrates a discrete second cutting block configured to perform a single cut—a sulcus cut, including a frame with a central cut-out portion, sizing indicia, anchoring holes, and an angled sulcus cutting guide including a ramp configured to guide a sulcus cutting tool. The discrete second cutting block can be deployed and attached onto the knee after the 4-in-1 cutting block is removed and the associated cuts made. FIG. 1M is a side view of the discrete cutting block of FIG. 1L. FIG. 1N illustrates a blade view of the cutting block of FIGS. 1L and 1M, showing the cutting tool after being advanced on the sulcus cutting guide ramp.

In some embodiments of the invention, the knee arthroplasty procedure as described, for example, above can proceed similarly until the application of a second cutting block to the distal femur that is different from the first cutting block. With use of some embodiments of 5-in-1 cutting blocks as disclosed herein, all cuts for the distal femur are completed with a single cutting block, and without the need to remove the first cutting block, and position a second cutting block configured to perform the sulcus cut, because the 5-in-1 cutting block is also configured for sulcus cutting. The combination cutting block, e.g., 5-in-1 cutting block can be the only block used for femur preparation as well as sulcus cutting in the TKA procedure. The 5-in-1 block can include a slot in the inferior surface connected to an oblique ramp extending superiorly and posteriorly within the housing of the block away from the anterior face of the 5-in-1 cutting block, and extending (e.g., completely extending) to the posterior (e.g., bone-engaging) face of the 5-in-1 cutting block, of which a sulcus cutting tool can extend therethrough. In some embodiments, the slot is generally symmetric with the midline of the inferior surface of the cutting block, and extends laterally from the midline in both directions as illustrated. The 5-in-1 block can be configured to perform exactly 5 cuts in some cases.

Advantages of the combination 5-in-1 cutting block include, for example, that the entire tray dedicated to the second cutting block (separate sulcus cutting block or guide) is no longer required, and the procedure can be completed quickly and more efficiently with an equivalent or better outcome. As such, some systems and methods do not involve a discrete single-cut sulcus cutting block or guide.

FIG. 2A illustrates an anterior perspective view of a combination 5-in-1 cutting block 200, according to some embodiments. The cutting block 200 can include, for example, any number of features as disclosed in the 4-in-1 cutting block of FIGS. 1A-1I, including an anterior cutting guide (e.g., slot), posterior cutting guide, and a plurality of chamfer cutting guides.

The cutting block 200 can additionally include one or more features 207 configured to perform a sulcus cut, thus creating a 5-in-1 cutting block in some embodiments. The cutting block 200 can include a superior end 202 and an inferior end 204, the inferior end 204 including a sulcus cutting slot 207 (e.g., cutting slot entrance) and a ramp guide (not shown) that can be partially or completely enclosed within the cutting block except for entrance and exit slots, and extends obliquely at an angle superiorly and posteriorly from the inferior end 204. The sulcus cutting slot 207 can extend laterally in both directions from the midline of the inferior end 204 as shown.

In some embodiments, the angle is about, for example, 25 degrees from vertical and/or from the plane of the anterior-facing surface (e.g., outer surface) of the cutting block 200, such as between about 15 degrees and about 35 degrees, or between about 20 degrees and about 30 degrees. In some embodiments, the angle is about 5, 10, 15, 20, 25, 30, 35, 40, 45, 50, 55, 60, 65, 70, 75, 80, or 85 degrees or more or less, or ranges including any two of the foregoing values.

FIG. 2B illustrates a posterior perspective view of the bone-engaging surface 220 of the cutting block of FIG. 2A, also illustrating the exit slot 203 of the sulcus cutting slot 207 and connected via a ramp guide 205 which can be partially or fully enclosed within the cutting block. The exit slot 203 can be relatively superior and posterior to the sulcus cutting slot 207 entrance as described, for example, above, and can extend laterally in both directions from the midline of bone-engaging surface 220 as shown.

FIG. 2C illustrates another view of the inferior surface of the cutting block, including the entrance sulcus cutting slot 207, which can have a width dimension 207A. The width dimension 207A can be, for example, between about 0.80 inches and about 1.25 inches, between about 0.90 inches and about 1.20 inches, about 0.80, 0.85, 0.90, 0.95, 1.00, 1.05, 1.10, 1.15, 1.20, 1.25 inches, or ranges including any two of the foregoing values. In some embodiments, the width dimension can be about 1.04 inches.

The slot 207 can be configured to allow a bone cut depth 211, for example, of between about 0.30 inches and about 0.60 inches, between about 0.35 inches and about 0.50 inches, about 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60 inches or more or less, and ranges including any two of the foregoing values.

In some embodiments, as illustrated in FIG. 2D, a low-profile cutting block 280 can have a reduced footprint in comparison to other cutting blocks as described and illustrated, for example, herein allowing improved visualization for the execution of saw cuts, increasing the accuracy of cuts and reducing the time required for preparation of the distal femur prior to prosthesis implantation. The cut-out portions can also, in some embodiments, advantageously allow for significant weight and material reduction in the cutting block, and also allow for an additional viewing channel. The reduced footprint can be created by, for example, voids or cut-out portions that include an open lateral end, and are not configured for bone cuts. The cut-out portions can be, for example, a plurality of laterally spaced-apart cut-out portions 222, 223 as illustrated extending from an outer surface of the cutting block, and separated by a bridge 226 connecting a more inferior portion of the cutting block to a superior support 221 including the anterior cutting slot 224. In other words, the cut-out portions 222, 223 can reside between the anterior cutting slot 224 and the chamfer cutting slot 236 in some embodiments. The cutting block 280 can, in some cases, be devoid of anterior referencing pin holes which have been replaced by the cut-out portions 222, 223. The superior support 221 can be configured for resting on top of a bone such as, for example, the knee during a portion of the orthopedic procedure.

A drill aperture 232 for sizing can be present in the anterior cutting slot 224, such as at the midline of the anterior cutting slot 224 for example, with a diameter greater than the maximal thickness of the anterior cutting slot 224 of the cutting block where the drill aperture 232 is not present, as shown. The drill aperture 232 can be configured to allow for a drill bit of a preset diameter used to confirm the proper sizing. In some embodiments, the drill aperture 232 can be configured to house a 1/16, ⅛, 3/16, ¼, 5/16, ⅜, 7/16, or ½ inch, or more or less drill bit therethrough, and ranges including any two of the foregoing values. In some embodiments, the drill aperture can have a sidewall pathway that is angled with respect to horizontal, the plane of the outer surface, and/or the anterior cutting slot. The angle can be in some cases between about 2 degrees and about 10 degrees, or about 5 degrees in some embodiments.

In some embodiments, the cut-out portions 223, 224 can, for example, collectively span at least about 50%, 60%, 70%, 75%, 80%, 85%, 90%, 95% or more or less of the width of the outer surface of the cutting block (and ranges including any two of the foregoing values), and separated only by the central bridge 226.

In some embodiments, the cut-out portions 223, 224 can, for example, collectively span at least about 5%, 10%, 15%, 20%, 25% or more or less of the total surface area of the outer surface of the cutting block (and ranges including any two of the foregoing values).

The cutting block 280 can include, for example, any number of features as disclosed in other embodiments of cutting blocks herein, including an anterior cutting guide (e.g., slot), posterior cutting guide, and a plurality of chamfer cutting guides. The cutting block can also include an integrated sulcus cutting guide (not shown in FIG. 2D—visualized in 3F) including a ramp or slot as disclosed elsewhere herein that can be partially or completely enclosed within the cutting block except for entrance and exit slots, and extends obliquely at an angle superiorly and posteriorly from the inferior end. The sulcus cutting slot can extend laterally in both directions from the midline of the inferior end. In some embodiments, the sulcus cutting slot does not extend inferiorly past the peripheral edge of the cutting block.

FIG. 2D1 illustrates an end view of the cutting block of FIG. 2D, including a sulcus cutting tool inserted through a sulcus cutting guide as described elsewhere herein.

FIGS. 2E and 2F include anterior/inferior angled and posterior angled respective perspective views of a combination cutting block 330, that can include any number of features as described elsewhere herein, including but not limited to an anterior cutting guide (e.g., slot), posterior cutting guide, and a plurality of chamfer cutting guides. The cutting block 330 can also include an oblique cutting ramp guide 340 configured to facilitate a sulcus cut. The oblique cutting ramp guide 340 can extend from the inferior surface of the cutting block, and extend at an angle posteriorly and superiorly as shown. In some embodiments, the angle is about, for example, 25 degrees from vertical, and/or from the plane of the anterior-facing surface (e.g., outer surface) of the cutting block 200, such as between about 15 degrees and about 35 degrees, or between about 20 degrees and about 30 degrees. In some embodiments, the angle is about 5, 10, 15, 20, 25, 30, 35, 40, 45, 50, 55, 60, 65, 70, 75, 80, or 85 degrees or more or less, or ranges including any two of the foregoing values.

FIG. 2G illustrates a cross-sectional view of a cutting block that could include, for example, features of the cutting block of FIGS. 2A-2B or others as disclosed elsewhere herein. The cutting block can include an oblique cutting ramp guide 205 configured to facilitate a sulcus cut. The oblique cutting ramp guide 205 can extend from an entrance slot 207 of the inferior surface 204 of the cutting block, and extend at an angle posteriorly and superiorly as shown to an exit slot 203 on the bone-engaging surface 220 of the cutting block. In some embodiments, the angle A is about, for example, 25 degrees from vertical, and/or from the plane of the anterior-facing surface (e.g., outer surface) of the cutting block 200, or other angles or ranges as disclosed herein. In some embodiments, the axial distance D from the inferior surface 204 of the cutting block to the exit slot 203 is about 0.60, 0.70, 0.75, 0.76, 0.77, 0.78, 0.79, 0.80, 0.81, 0.82, 0.83, 0.84, 0.85, 0.86, 0.87, 0.88, 0.89. 0.90, 0.91, 0.92, 0.93, 0.94, 0.95, 0.96, 0.97, 0.98, 0.99, 1.00, 1.05, 1.10, 1.15, 1.20, 1.25, 1.30, 1.40, 1.50 inches or more or less, or ranges including any two of the foregoing values.

FIGS. 3A-3G illustrate sequences of a method of performing a total knee arthroplasty utilizing a combination 5-in-1 cutting block, according to some embodiments of the invention. FIG. 3A illustrates a perspective view of an embodiment of the cutting block of FIGS. 2D-2D1 placed on a sawbones knee model, with fixation pins placed in the guide holes, e.g., posterior referencing pin holes for anchoring to the knee. FIG. 3B illustrates a front view of the cutting block of FIG. 3A on the knee model. FIG. 3C illustrates an angled side view, with a cutting tool inserted through the anterior cutting slot for making an anterior cut. FIG. 3D illustrates a top view of FIG. 3C. FIG. 3E illustrates a perspective view of a cutting tool inserted into a chamfer cutting guide, for making an anterior chamfer cut. FIG. 3F illustrates a view of a cutting tool inserted into the sulcus cutting slot of the combination 5-in-1 cutting lock, which as noted above advantageously can occur without the need to replace a first cutting block with a second cutting block. FIG. 3G illustrates another view of the cutting tool inserted into the sulcus cutting guide of FIG. 3F. The cutting tool can be then inserted completely through the sulcus cutting guide, such that the distal end of the cutting tool emerges past the exit slot of the sulcus cutting guide of the 5-in-1 cutting block.

FIG. 3H illustrates a discrete sulcus cutting block (upper left); 4-in-1 cutting block (upper right), and a low-profile combination 5-in-1 cutting block (bottom), each of which can be as previously described.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “performing a sulcus cut utilizing an integrated 5-in-1 cutting block” includes “instructing performing a sulcus cut utilizing an integrated 5-in-1 cutting block.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1. A method of performing a knee arthroplasty procedure using no more than a single cutting block to prepare a femur, comprising:
securing a single cutting block with respect to the distal femur;
resecting a portion of the anterior femur utilizing the cutting block;
resecting a portion of the posterior condyle utilizing the cutting block;
resecting a portion of the anterior chamfer utilizing the cutting block;
resecting a portion of the posterior chamfer utilizing the cutting block; and
resecting a portion of the sulcus utilizing the cutting block without removing the cutting block after resecting a portion of the posterior chamfer utilizing the cutting block.

2. The method of claim 1, further comprising removing the cutting block after resecting a portion of the sulcus.

3. The method of claim 1, further comprising positioning a knee prosthesis over the resected portions of the knee.

4. The method of claim 1, wherein resecting a portion of the sulcus comprises inserting a sulcus cutting tool in a sulcus cutting guide on an inferior surface of the cutting block.

5. The method of claim 4, wherein the sulcus cutting guide comprises a ramp extending superiorly and posteriorly from the inferior surface of the cutting block.

6. The method of claim 4, further comprising inserting the sulcus cutting tool in a slot on the inferior surface.

7. The method of claim 1, further comprising inserting a drill into a central aperture in the anterior cutting guide to confirm sizing.

8. The method of claim 1, wherein securing the single cutting block with respect to the distal femur comprises inserting a plurality of pins into posterior referencing pin holes in the single cutting block.

9. An integrated, combination cutting block for preparing a femur during a knee arthroplasty procedure, comprising:
an outer-facing surface, a bone-engaging surface, a superior surface, and an inferior surface;
an anterior cutting guide;
a posterior cutting guide;
a plurality of chamfer cutting guides; and
a sulcus cutting guide inferior to the anterior, posterior, and chamfer cutting guides.

10. The combination cutting block of claim 9, wherein the sulcus cutting guide comprises a guide ramp that extends from the inferior surface posteriorly and superiorly to at least the outer-facing surface, and at an angle with respect to a plane of the outer-facing surface.

11. The combination cutting block of claim 10, wherein the sulcus cutting guide is a captured cutting guide.

12. The combination cutting block of claim 11, wherein the sulcus cutting guide comprising an entrance slot on the inferior surface and an exit slot on the bone-engaging surface.

13. The combination cutting block of claim 10, wherein the sulcus cutting guide is a non-captured cutting guide.

14. The combination cutting block of claim 10, wherein the angle is between about 15 degrees and about 35 degrees.

15. The combination cutting block of claim 14, wherein the angle is about 25 degrees.

16. A low-profile cutting block for preparing a femur during a knee arthroplasty procedure, comprising:
an outer-facing surface, a bone-engaging surface, a superior surface, and an inferior surface;
an anterior cutting guide;
a posterior cutting guide;
a plurality of chamfer cutting guides; and
a plurality of laterally spaced apart cut-out portions between the anterior cutting guide and plurality of chamfer cutting guides, each laterally spaced apart cut-out portions spaced apart by a bridge.

17. The cutting block of claim 16, wherein the anterior cutting guide comprises a slot, and the slot comprises a drill aperture comprising a diameter greater than the width of the slot.

18. The cutting block of claim 16, wherein a surface area of the cut-out portions is between about 5% and about 25% of the total surface area of the outer-facing surface.

19. The cutting block of claim 16, further comprising a sulcus cutting guide on the cutting block positioned inferior to the anterior, posterior, and chamfer cutting guides.

20. The cutting block of claim 16, wherein the cutting block does not comprise anterior referencing pin holes.

21. (canceled)

22. (canceled)
